(19) United States
(12) Patent Application Publication
(10) Pub. No.: US 2026/0194528 A1
(43) Pub. Date: Jul. 9, 2026

(54) PROXIMITY-BASED LABELING OF BIOMOLECULAR CONDENSATE MICROENVIRONMENTS

(71) Applicant: THE TRUSTEES OF PRINCETON UNIVERSITY, Princeton, NJ (US)

(72) Inventors: David W.C. MacMillan, Princeton, NJ (US); Chenmengxiao Pan, Princeton, NJ (US); Steven Douglas Knutson, Princeton, NJ (US); Sean Huth, Princeton, NJ (US)

(21) Appl. No.: 19/128,844

(22) PCT Filed: Nov. 13, 2023
(86) PCT No.: PCT/US2023/079482

§ 371 Date: May 9, 2025

Related U.S. Application Data
(60) Provisional application No. 63/424,581, filed on Nov. 11, 2022.

(51) Int. Cl. G01N 33/58; B01J 31/18; B01J 31/22; B01J 35/39

(52) U.S. Cl. CPC G01N 33/58; B01J 31/1815; B01J 31/2295; B01J 35/39; B01J 2531/827; G01N 2333/914

(57) ABSTRACT

Composites, systems and methods are described herein enabling the profiling of local microenvironments across various biomolecular condensates via proximity labeling. In some embodiments, a composite includes one or more biomolecular condensates comprising nucleic acids and/or functionally diverse proteins, and a transition metal catalyst coupled to the biomolecular condensate via interaction with at least one of the nucleic acids or functionally diverse proteins. Composites described herein, in some embodiments, are located in the intracellular environment.

### RELATED APPLICATION DATA

The present application claims priority pursuant to Article 8 of the Patent Cooperation Treaty to U.S. Provisional Patent Application Ser. No. 63/424,581 filed Nov. 11, 2022 which is hereby incorporated by reference in its entirety.

### FIELD

The present invention relates to compositions, systems, and methods for proximity-based labeling and, in particular, for proximity-based labeling to profile local microenvironments of various biomolecular condensates.

### BACKGROUND

Protein proximity labeling has emerged as a powerful approach for profiling protein interaction networks. The ability to label associated or bystander proteins through proximity labeling can have important implications on further understanding the cellular environment and biological role of a protein of interest. Current proximity labeling methods all involve the use of enzyme-generated reactive intermediates that label neighboring proteins on a few select amino acid residues through diffusion or physical contact. Despite the transformative impact of this technology, the inherent stability of these reactive intermediates such as phenoxy radicals (t_{1/2}>100 μs) through peroxidase activation or biotin-AMP (t_{1/2}>60 s) through biotin ligases can promote diffusion far from their point of origin. As a result, these enzyme-generated reactive intermediates pose a challenge to profiling within tight micro-environments. Furthermore, the large enzyme size, the dependency on certain amino acids for labeling, and the inability to temporally control these labeling systems present additional challenges for profiling within confined spatial regions. Given these limitations, new approaches for proximity-based labeling are needed.

### SUMMARY

In view of the foregoing disadvantages, composites, systems and methods are described herein enabling the profiling of local microenvironments across various biomolecular condensates via proximity labeling. Biomolecular condensates are compartments in eukaryotic cells which concentrate biomolecular species, including nucleic acids and proteins. In contrast to organelles, biomolecular condensates lack surrounding membranes. Liquid-liquid phase separation can drive formation of various biomolecular condensates, wherein various biomolecules aggregate via intra- and intermolecular interactions.

In some embodiments, a composite includes one or more biomolecular condensates comprising nucleic acids and/or functionally diverse proteins, and a transition metal catalyst coupled to the biomolecular condensate via interaction with at least one of the nucleic acids or functionally diverse proteins. Composites described herein, in some embodiments, are located in the intracellular environment. In some embodiments, for example, a composite is located in the nucleus of the cell. Alternatively, composites can be located in the cytoplasm or membranes of the cell. The transition metal catalyst can interact with a nucleic acid, such as RNA, or a protein of the biomolecular condensate by covalent bonding. In other embodiments, the transition metal catalyst may interact with a nucleic acid or protein of the condensate via electrostatic interactions and/or van der Waals interactions.

Composites described herein can perform various cellular functions depending on composition and location of the composites. In some embodiments, biomolecular condensates of composites described herein are selected from the group consisting of P-bodies, U-bodies, stress granules, centrosomes, signaling clusters, membrane clusters, synaptic densities, RNA transport granules, Balbiani bodies, germ granules, nuclear speckles, OPT domains, gems, PcG bodies, Cajal bodies, perinucleolar compartments, cleavage bodies, and PML bodies. Types and locations of composites can be specific to specific cell types. For example, RNA transport granules and synaptic densities are found in neuronal cells, while Balbani bodies and germ granules are found in germ cells.

The transition metal catalyst coupled to the biomolecular condensate can comprise a platinum group metal center, in some embodiments. Moreover, in some embodiments, the transition metal catalyst is of Formula I:

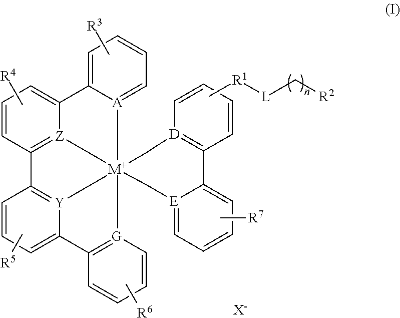

wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl; wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20. As provided in Formula I, the linking moiety, L, is optional and, therefore, may not be present in some embodiments of the transition metal catalyst.

Polarity of the transition metal complexes can be tailored to specific cellular environments via selection of R³-R⁷. In some embodiments, for example, one or more of R³-R⁷ are selected to exhibit hydrophobic, lipophilic, or non-polar character. In some embodiments, for example, one or more of R³-R⁷ can be alkyl, fluoro, or fluoroalkyl. Transition metal complexes described herein exhibiting hydrophobic, lipophilic, or non-polar character can be suitable for placement or passage into intracellular environments, including intracellular biomolecular condensate environments. The transition metal complexes can pass through the cellular membrane for mapping local intracellular environments according to the principles described herein. Accordingly, such transition metal complexes are cell permeable. Alternatively, R³-R⁷ are selected to exhibit hydrophilic character via charged and/or polar chemical moieties. In such embodiments, the transition metal complex can exhibit hydrophilic character suitable for placement in intercellular/extracellular environments.

As described further herein, the transition metal catalyst can have electronic structure for energy transfer to a protein labeling agent to produce a reactive intermediate. In some embodiments, the energy transfer is Dexter energy transfer or electron transfer, including single electron transfer. Energy transfer to the protein labeling agent can originate from an excited state of the transition metal catalyst electronic structure, in some embodiments. The excited state of the catalyst, for example, can be a singlet excited state or triplet excited state. The excited state of the catalyst can be generated by one or more mechanisms, including energy absorption by the catalyst. In some embodiments, the catalyst is a photocatalyst, wherein the excited state is induced by absorption of one or more photons. In other embodiments, the catalyst may be placed in an excited state by interaction with one or more chemical species in the surrounding environment. Alternatively, energy transfer to the protein labeling agent, including electron transfer, may originate from a ground state of the catalyst electronic structure. Energy transfer the protein labeling agent to produce the reactive intermediate permits profiling of microenvironments local to biomolecular condensates, as described herein.

In another aspect, systems for profiling microenvironments local to biomolecular condensates are provided. A system, in some embodiments, comprises a protein labeling agent, and a composite, the composite including one or more biomolecular condensates comprising nucleic acids and/or functionally diverse proteins, and a transition metal catalyst coupled to the biomolecular condensate via interaction with at least one of the nucleic acids or functionally diverse proteins, wherein the transition metal catalyst has electronic structure permitting energy transfer to the protein labeling agent to provide a reactive intermediate. As described herein, the biomolecular condensate can be located within the nucleus, cytoplasm, or membrane(s) of the cell. The reactive intermediate is operable to label a protein or other biomolecule within a predetermined radius of the composite. The predetermined radius may be a diffusion radius of the reactive intermediate.

The diffusion radius of the reactive intermediate can be tailored to specific microenvironment mapping (proximity-based labeling) considerations, and can be limited to the nanometer scale. In some embodiments, for example, the diffusion radius of the reactive intermediate can be less than 100 nm, less than 50 nm, less than 10 nm, less than 5 nm, less than 4 nm, less than 3 nm, or less than 2 nm prior to quenching in the surrounding environment. The diffusion radius can be 0.5 nm to 10 nm or 0.5 nm to 100 nm, in some embodiments. Accordingly, the reactive intermediate will react or crosslink with a protein or other biomolecule within the diffusion radius or be quenched by the surrounding environment if no protein or biomolecule is present. In this way, high resolution of the environment local to the biomolecular condensate can be mapped via concerted effort between the transition metal catalyst and protein labeling agent. Additionally, the reactive intermediate can exhibit a t_{1/2} less than 5 ns, less than 4 ns, or less than 2 ns prior to quenching, in some embodiments. The reactive intermediate, for example, can exhibit a t_{1/2} less of 1-5 ns. In additional embodiments, the diffusion radius can be extended to between 5-500 nm though extension of the reactive intermediate half-life. For example, in some embodiments, the reactive intermediate can have a half-life of 1-100 μs, or greater.

In another aspect, methods of profiling microenvironments local to biomolecular condensates are described herein. A method comprises forming a composite comprising a transition metal catalyst coupled to a biomolecular condensate via interaction with a biomolecular species of the condensate, and activating a protein labeling agent to a reactive intermediate with the transition metal catalyst. The reactive intermediate is coupled to a protein or other biomolecule within a predetermined radius of the composite. The biomolecular condensate, transition metal catalyst, protein labeling agent, and reactive intermediate can have any composition and/or properties described herein. Moreover, the transition metal catalyst can be coupled to the biomolecular condensate via covalent bonding, electrostatic interactions or van der Waal interactions. In some embodiments, the protein labeling agent is activated at differing times in the biomolecular condensate lifecycle, including early disassembly, mid-disassembly, and late disassembly. Additionally, methods described herein further comprise detecting or analyzing the protein or other biomolecule coupled to the reactive intermediate.

These and other embodiments are further described in the following detailed description.

### BRIEF DESCRIPTION OF THE DRAWINGS

FIG. 1 illustrates transition metal catalysts described herein according to some embodiments.

FIG. 2 illustrates binding of a transition metal complex described herein to a protein via haloalkane dehalogenase, according to some embodiments.

FIG. 3 illustrates activation of protein labeling agent to map key proteins of interest over a biomolecular condensate lifecycle, including early disassembly, mid-disassembly, and late disassembly, according to some embodiments.

FIG. 4 is a schematic detailing stress granule interactome mapping with composite and protein labeling agent described herein, according to some embodiments.

FIG. 5 illustrates ubiquitination of stress granule proteins during granule formation.

FIG. 6 illustrates protein ubiquitination is undermined by the presence of TAK243 during granule formation and disassembly.

FIG. 7 is a volcano plot identifying HECT ubiquitin ligases and autography adaptors identified by the stress granule interactome mapping with composite and protein labeling agent described herein, according to some embodiments.

FIG. 8 provides time resolution of the HECT E3 ligases recruited the stress granules as determined by interactome mapping with composite and protein labeling agent described herein, according to some embodiments.

FIG. 9 is a schematic for employing Heclin to determine effects on HECT E3 ligase function relative to stress granule disassembly.

FIG. 10 illustrates HECT E3 ligase inhibition with Heclin delays stress granule disassembly.

FIG. 11 evidences that HECT E3 ligase siRNA knockdown delays stress granule disassembly.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents.

The term “alkynyl” as used herein, alone or in combination, refers to a straight or branched chain hydrocarbon group having at least one carbon-carbon triple bond and optionally substituted with one or more substituents.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, boron, oxygen and/or sulfur.

The term “heterocycle” as used herein, alone or in combination, refers to an mono- or multicyclic ring system in which one or more atoms of the ring system is an element other than carbon, such as boron, nitrogen, oxygen, and/or sulfur or phosphorus and wherein the ring system is optionally substituted with one or more ring substituents. The heterocyclic ring system may include aromatic and/or non-aromatic rings, including rings with one or more points of unsaturation.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as boron, nitrogen, oxygen, sulfur or phosphorus, alone or in combination, and wherein the ring system is optionally substituted with one or more ring substituents.

The term “alkoxy” as used herein, alone or in combination, refers to the moiety RO—, where R is alkyl, alkenyl, or aryl defined above.

The term “halo” as used herein, alone or in combination, refers to elements of Group VIIA of the Periodic Table (halogens). Depending on chemical environment, halo can be in a neutral or anionic state.

Terms not specifically defined herein are given their normal meaning in the art.

### I. Composites

In one aspect, composites are described herein enabling the profiling of local microenvironments across biomolecular condensates via proximity labeling. In some embodiments, a composite includes one or more biomolecular condensates comprising nucleic acids and/or functionally diverse proteins, and a transition metal catalyst coupled to the biomolecular condensate via interaction with at least one of the nucleic acids or functionally diverse proteins. Composites described herein, in some embodiments, are located in the intracellular environment. In some embodiments, for example, a composite is located in the nucleus of the cell. Alternatively, composites can be located in the cytoplasm or membranes of the cell. The transition metal catalyst can interact with a nucleic acid, such as RNA, or a protein of the biomolecular condensate by covalent bonding. In other embodiments, the transition metal catalyst may interact with a nucleic acid or protein of the condensate via electrostatic interactions and/or van der Waals interactions.

The transition metal complex, in some embodiments, is coupled to a protein of the biomolecular condensate via interaction with haloalkane dehalogenase (HaloTag). Haloalkane dehalogenase can be co-expressed with the protein using standard cloning procedures. The transition metal complex can be functionalized with a haloalkane moiety for covalently binding with the haloalkane dehalogenase resulting in coupling of the protein to the biomolecular condensate. FIG. 2 illustrates binding of a transition metal complex described herein to a protein via haloalkane dehalogenase, according to some embodiments. Alternatively, the transition metal complex can be bonded to a derivatized amino acid of a biomolecular condensate protein. Suitable click chemistry moieties of the transition metal complex and/or derivatized amino acid can be selected from the group consisting of DBCO, BCN, TCO, tetrazine, alkyne and azide. The transition metal complex can be of Formula (I) herein. In other embodiments, the transition metal complex is coupled to a protein of a biomolecular condensate via protein-trans splicing, the transition metal complex being initially coupled to a split intein. The spit intein carrying the transition metal complex can be an N-intein or an C-intein.

In some embodiments, the transition metal catalyst is of Formula I:

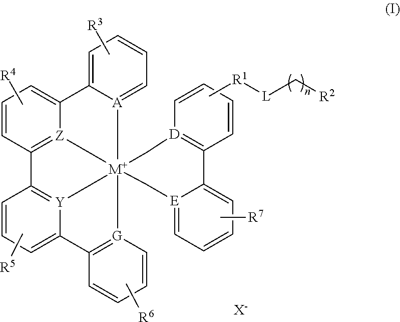

wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl; wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20. As provided in Formula I, the linking moiety, L, is optional and, therefore, may not be present in some embodiments of the transition metal catalyst.

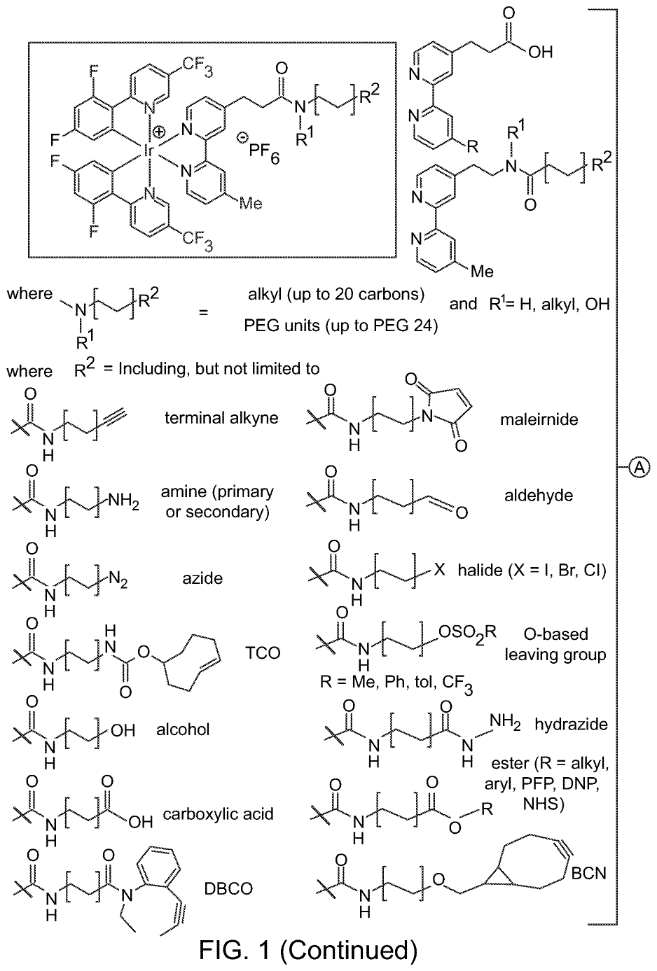

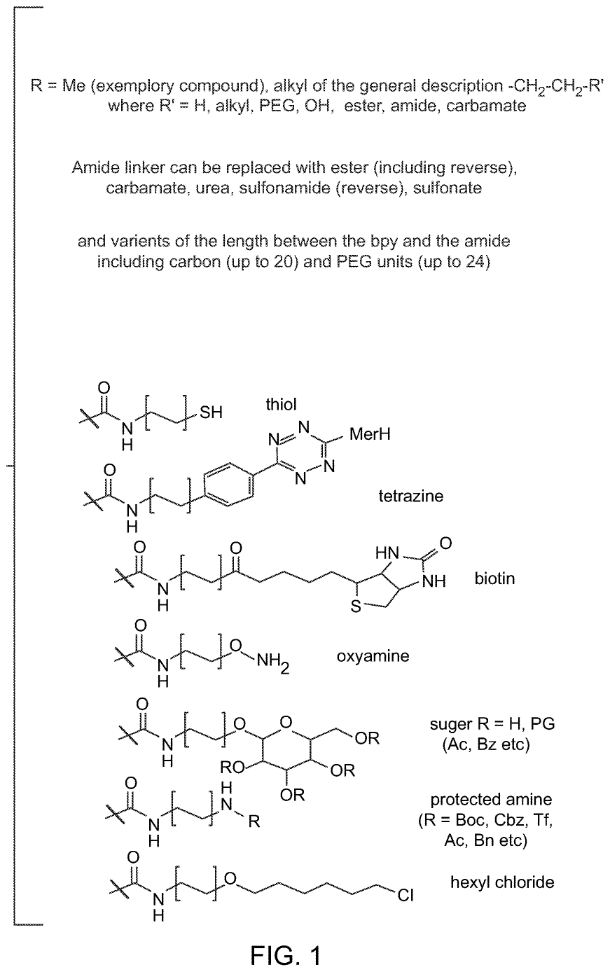

It is understood that hydrogen occupies positions on the aryl rings of Formula I in the absence of optional substituents R³-R⁷. Additionally, in some embodiments, counterion (X) can be selected from tetraalkylborate, tetrafluoroborate, tetraphenylborate, PF₆⁻, and chloride. Polarity of the transition metal complexes can be tailored to specific cellular environments via selection of R³-R⁷. In some embodiments, for example, one or more of R³-R⁷ are selected to exhibit hydrophobic, lipophilic, or non-polar character. In some embodiments, for example, one or more of R³-R⁷ can be alkyl, fluoro, or fluoroalkyl. Transition metal complexes described herein exhibiting hydrophobic, lipophilic, or non-polar character can be suitable for placement or passage into intracellular environments. The transition metal complexes can pass through the cellular membrane for mapping biomolecular condensates in the intracellular environments according to the principles described herein. Accordingly, such transition metal complexes are cell permeable. FIG. 1 illustrates various transition metal complexes described herein. In some embodiments, L is an amide in conjunction with a polyethylene glycol (PEG) moiety for connection to R². As provided in FIG. 1, the PEG moiety can be replaced with an alkylene moiety. In some embodiments, for example, R² comprises a haloalkane or a click chemistry moiety including, but not limited to, BCN, DBCO, TCO, tetrazine, alkyne, and azide. As illustrated in FIG. 1, these click chemistries of R² can be directly coupled to the linker (L) or coupled via a heteroatom, aryl, or carbonyl.

The transition metal catalyst can have electronic structure for energy transfer to a protein labeling agent to produce a reactive intermediate. In some embodiments, the energy transfer is Dexter energy transfer or electron transfer. Energy transfer to the protein labeling agent can originate from an excited state of the transition metal catalyst electronic structure, in some embodiments. The excited state of the catalyst, for example, can be a singlet excited state or triplet excited state. A transition metal complex of Formula I, in some embodiments, can exhibit a long-lived triplet excited state (T₁) facilitating energy transfer to the protein labeling agent.

The T₁ state can have t_{1/2} of 0.2-2 μs, for example. Transition metal complexes described herein can be photocatalytic and, in some embodiments, absorb light in the visible region or infrared region of the electromagnetic spectrum. Absorption of electromagnetic radiation can excite the transition metal complex to the Si state followed by quantitative intersystem crossing to the T₁ state. The transition metal catalyst can subsequently undergo short-range Dexter energy transfer to a protein labeling agent, and returned to the ground state, S₀. The energy transfer to the labeling agent activates the labeling agent for reaction with a protein or other biomolecule in the local biomolecular condensate environment. The T₁ state of the transition metal complex can be greater than 60 kcal/mol, in some embodiments. The metal center, for example, can be selected from transition metals of the platinum group. The metal center can be iridium, in some embodiments.

In other embodiments, the catalyst may be placed in an excited state by interaction with one or more chemical species in the surrounding environment. Alternatively, energy transfer to the protein labeling agent, including electron transfer, may originate from a ground state of the catalyst electronic structure. Energy transfer the protein labeling agent to produce the reactive intermediate permits profiling of microenvironments local to biomolecular condensates, as described herein.

### II. Systems for Profiling Interactome of Biomolecular Condensate

In another aspect, systems for profiling microenvironments local to biomolecular condensates are provided. A system, in some embodiments, comprises a protein labeling agent, and a composite, the composite including one or more biomolecular condensates comprising nucleic acids and/or functionally diverse proteins, and a transition metal catalyst coupled to the biomolecular condensate via interaction with at least one of the nucleic acids or functionally diverse proteins, wherein the transition metal catalyst has electronic structure permitting energy transfer to the protein labeling agent to provide a reactive intermediate. The transition metal catalyst can be of Formula I and can have any composition, structure, and/or properties described in Section I hereinabove. The biomolecular condensate can be located within the nucleus, cytoplasm, or membrane(s) of the cell. The reactive intermediate is operable to label a protein or other biomolecule within a predetermined radius of the composite. The predetermined radius may be a diffusion radius of the reactive intermediate.

The diffusion radius of the reactive intermediate can be tailored to specific microenvironment mapping (proximity-based labeling) considerations, and can be limited to the nanometer scale. In some embodiments, for example, the diffusion radius of the reactive intermediate can be less than 100 nm, less than 50 nm, less than 10 nm, less than 5 nm, less than 4 nm, less than 3 nm, or less than 2 nm prior to quenching in the surrounding environment. The diffusion radius can be 0.5 nm to 10 nm, in some embodiments. Accordingly, the reactive intermediate will react or crosslink with a protein or other biomolecule within the diffusion radius or be quenched by the surrounding environment if no protein or biomolecule is present. In this way, high resolution of the environment local to the biomolecular condensate can be mapped via concerted effort between the catalyst and protein labeling agent. Additionally, the reactive intermediate can exhibit a t_{1/2} less than 5 ns, less than 4 ns, or less than 2 ns prior to quenching, in some embodiments. The reactive intermediate, for example, can exhibit a t_{1/2} less of 1-5 ns. In additional embodiments, the diffusion radius can be extended to between 5-500 nm though extension of the reactive intermediate half-life. For example, in some embodiments, the reactive intermediate can have a half-life of 1-100 μs, or greater. In some embodiments, more than one species of type of protein labeling agent can be employed in systems described herein. For example, two or more species having different diffusion radii can be employed in a system. In such embodiments, the differing diffusion radii can permit labeling at differing distances from the transition metal complex, thereby further mapping the interactome of the biomolecular condensate.

In some embodiments, the protein labeling agent can be a diazirine. Triplet energy transfer from the excited state photocatalyst can promote the diazirine to its triplet (T₁) state. The diazirine triplet under-goes elimination of N₂ to release a free triplet carbene, which undergoes picosecond-timescale spin equilibration to its reactive singlet state (t_{1/2}<1 ns) which either crosslinks with a nearby protein or is quenched in the aqueous environment. In some embodiments, the extinction coefficient of the transition metal complex is 3 to 5 orders of magnitude greater than that of the diazirine.

Any diazirine consistent with the technical principles discussed herein. Diazirine sensitization, for example, can be extended to a variety of p- and m-substituted aryltrifluoromethyl diazirines bearing valuable payloads for microscopy and proteomics applications, including free carboxylic acid, phenol, amine, alkyne, carbohydrate, and biotin groups. The diazirine can be functionalized with a marker, such as biotin. In some embodiments, the marker is desthiobiotin. The marker can assist in identification of proteins labeled by the protein labeling agent. The marker, for example, can be useful in assay results via western blot and/or other analytical techniques. Markers can include alkyne, azide, FLAG tag, fluorophore, and chloroalkane functionalities, in addition to biotin and desthiobiotin.

In additional embodiments wherein the transition metal catalyst is a photocatalyst, the protein labeling agent can be an azide. Triplet energy transfer from the excited state photocatalyst can promote nitrene formation from the azide. The reactive nitrene either crosslinks with a nearby protein or is quenched in the aqueous environment. Any azide operable to undergo energy transfer with eth transition metal photocatalyst for nitrene formation can be employed. In some embodiments, an azide is an aryl azide.

### III. Methods of Profiling Biomolecular Condensate Microenvironments

In another aspect, methods of profiling microenvironments local to biomolecular condensates are described herein. A method comprises forming a composite comprising a transition metal catalyst coupled to a biomolecular condensate via interaction with a biomolecular species of the condensate, and activating a protein labeling agent to a reactive intermediate with the transition metal catalyst. The reactive intermediate is coupled to a protein or other biomolecule within a predetermined radius of the composite. The biomolecular condensate, transition metal catalyst, protein labeling agent, and reactive intermediate can have any composition and/or properties described herein in Sections I and II above. Moreover, the transition metal catalyst can be coupled to the biomolecular condensate via covalent bonding, electrostatic interactions or van der Waal interactions, as described in Section I above. In some embodiments, the protein labeling agent is activated at differing times in the biomolecular condensate lifecycle, including early disassembly, mid-disassembly, and late disassembly. In some embodiments, the same protein labeling agent can be used over the condensate lifecycle. Alternatively, multiple different protein labeling agents can be used over the condensate lifecycle. The differing protein labeling agents can have similar or different diffusion radii for providing complete mapping of the biomolecular condensate interactome.

Additionally, methods described herein further comprise detecting or analyzing the protein or other biomolecule coupled to the reactive intermediate.

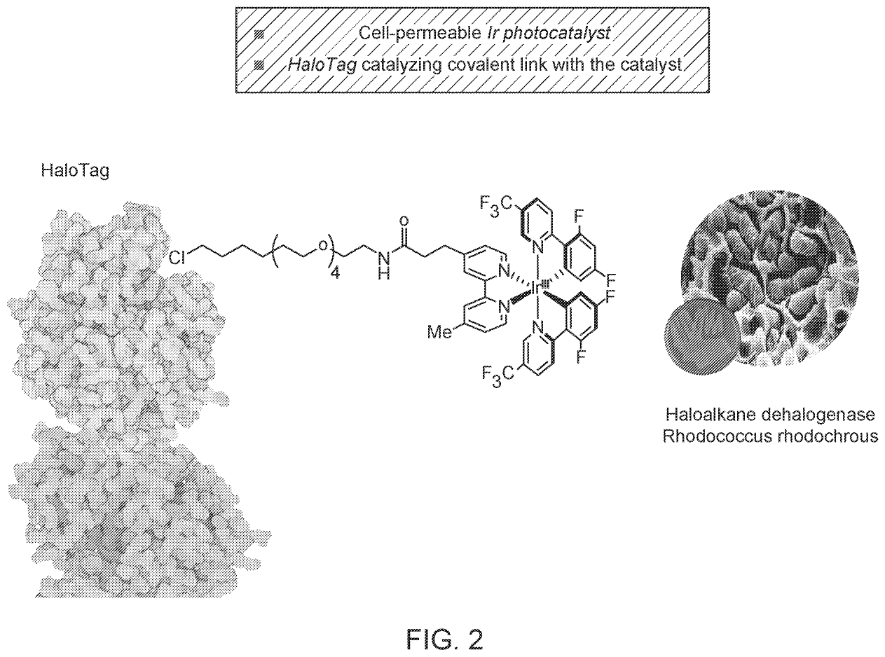

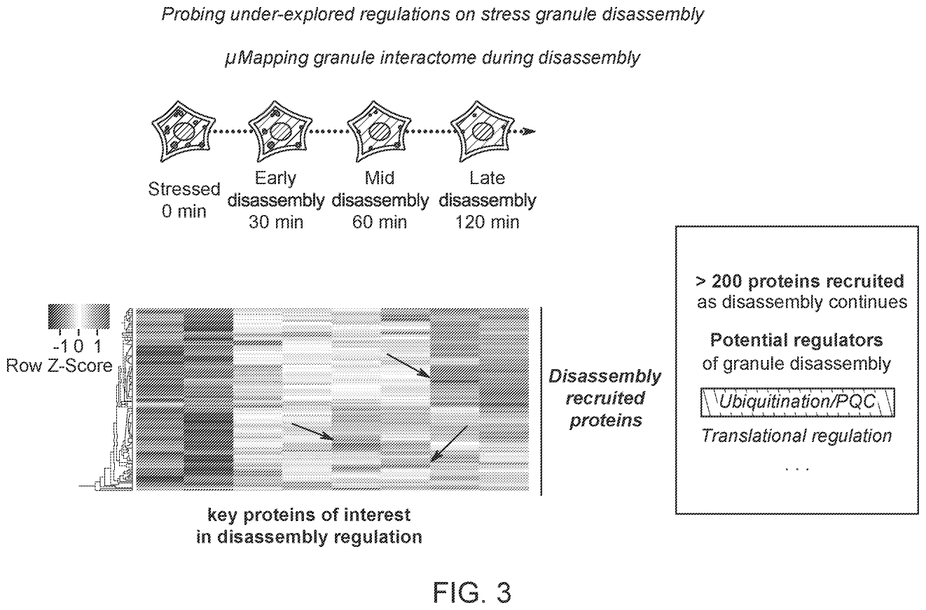

FIG. 3 illustrates activation of protein labeling agent over the biomolecular condensate lifecycle, including early disassembly, mid-disassembly, and late disassembly. The staged activation of the protein labeling agent with the transition metal complex of the composite can determine key proteins of interest in disassembly regulation of the condensate.

These and other embodiments are further illustrated in the following non-limiting examples.

### Example 1—Stress Granule Interactome Mapping

A protein-transition metal complex composite of FIG. 2 was prepared as follows. Cells (HEK243T stably expressing HaloTag-G3BP1) were seeded and grown to 95% confluency prior to labeling. The cells were incubated in DMEM media containing 5 μM of the with iridium catalyst of FIG. 2 for one hour. During this period, the iridium catalyst passes through the cell membranes and conjugates with the HaloTag-G3BP1 protein. The cells were then incubated in fresh DMEM media for one hour to remove or minimize noise from any unbound iridium catalyst.

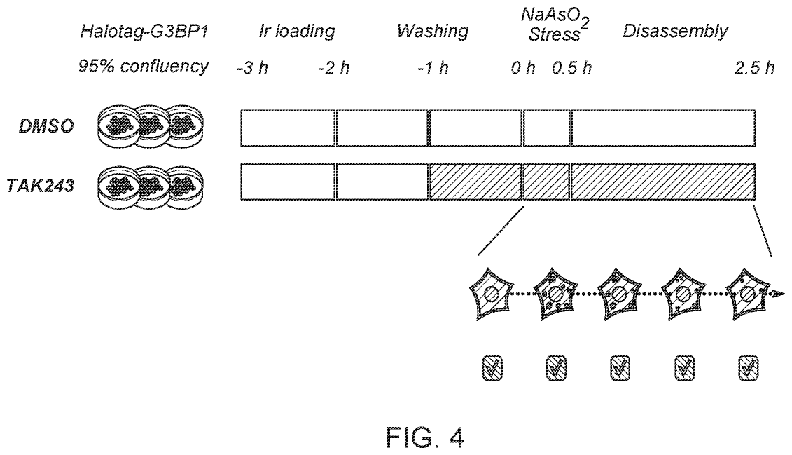

A first portion of the Ir-catalyst loaded cells were subsequently transferred to DMEM media pre-dosed with 2 μM TAK243 and incubated for another hour. The remaining second portion (control) was dosed with the same volume of DMSO. The cells were stressed with 500 mM NaAsO₂ in DMEM for 30 minutes. Following stressing, the media was replaced with fresh DMEM for cell recovery at the indicated time. During the stressing period, the cells were dosed with protein labeling agent of 500 μM biotin-diazirine in the media 30 minutes prior to the individual time point of photolabeling. During photolabeling, the cells were subjected to 10 minutes of 450 nm blue light irradiation. FIG. 4 illustrates the foregoing testing protocol.

General proteomics preparation protocol was followed. 600 μg of total protein was loaded for streptavidin enrichment. After on-bead denaturing, reduction, alkylation, and washes, biotin-labeled proteins are digested with trypsin to yield peptide solution for TIMSTOF LCMS analysis.

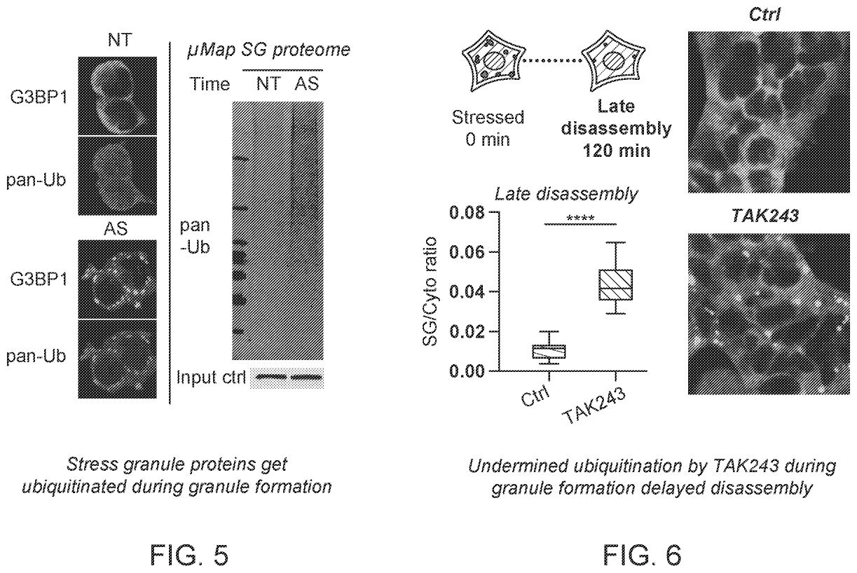

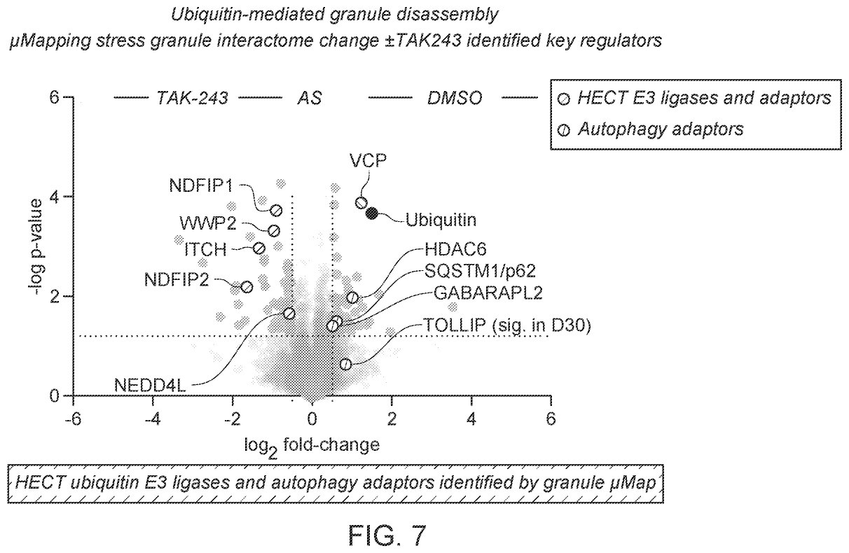

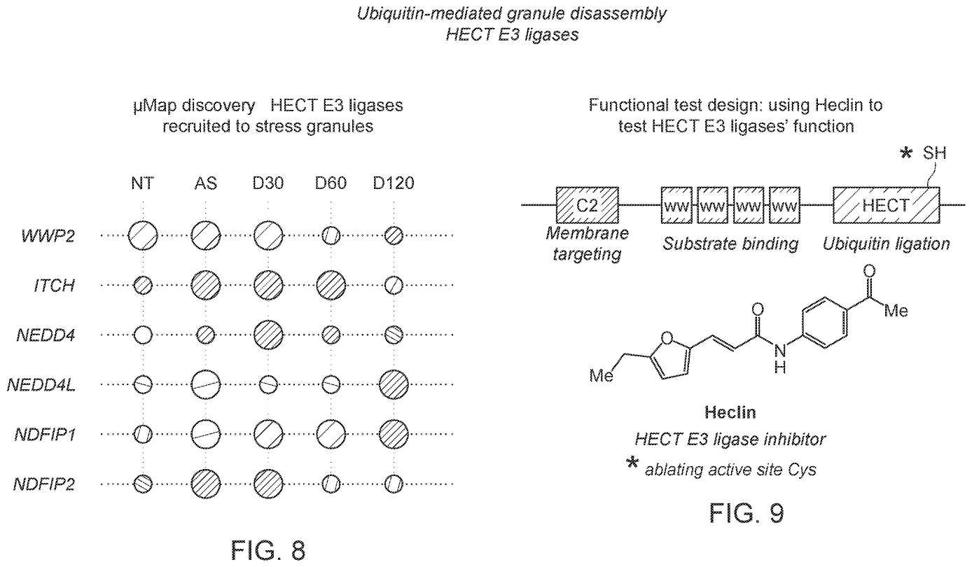

FIG. 5 illustrates ubiquitination of stress granule proteins during granule formation. Moreover, FIG. 6 illustrates when protein ubiquitination is undermined or inhibited by the presence of TAK243 during granule formation, granule disassembly will be delayed. FIG. 7 is a volcano plot identifying HECT ubiquitin ligases and autography adaptors identified in the stress granule interactome mapping with the composite and protein labeling agent of the present example. FIG. 8 provides time resolution of the HECT E3 ligases recruited to the stress granules.

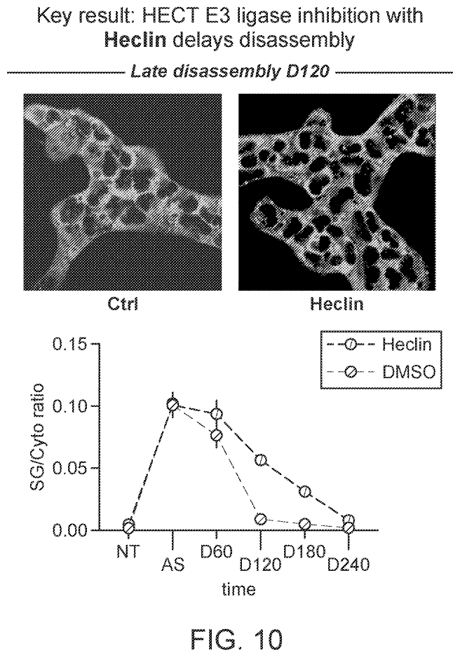

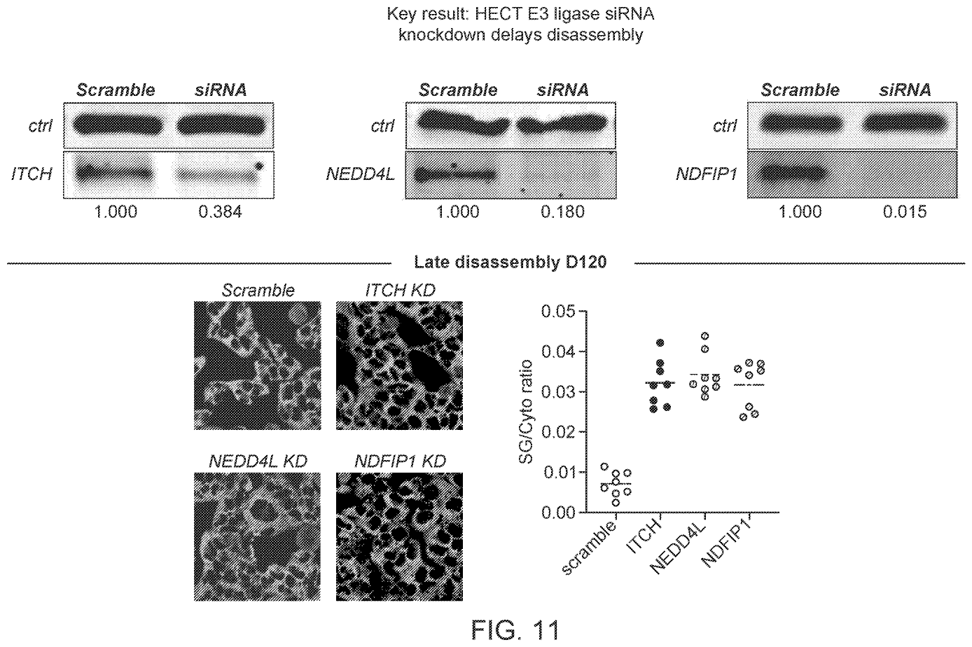

With this information provided by the interactome mapping, E3 ligase inhibitor Heclin was employed to determine any effects on HECT E3 ligase function relative to stress granule disassembly, as illustrated in FIG. 9. FIG. 10 illustrates HECT E3 ligase inhibition with Heclin delays stress granule disassembly. Additionally, in FIG. 11, HECT E3 ligase siRNA knockdown delays stress granule disassembly.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A composite comprising:
one or more biomolecular condensates comprising nucleic acids and/or functionally diverse proteins; and
a transition metal catalyst coupled to the biomolecular condensate via interaction with at least one of the nucleic acids or functionally diverse proteins.

2. The composite of claim 1, wherein the interaction is selected from the group consisting of a covalent bond, electrostatic interaction(s), and van der Walls interactions.

3. The composite of claim 1, wherein the transition metal complex is of the formula: 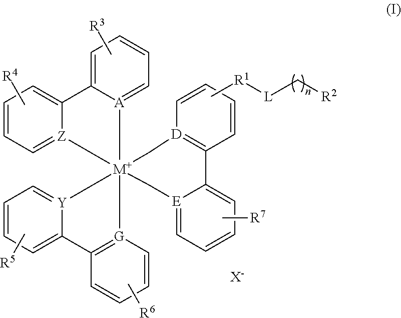
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

4. The composite of claim 3, wherein the transition metal complex is coupled to a protein of the biomolecular condensate via binding with haloalkane dehalogenase.

5. The composite of claim 3, wherein Mis iridium.

6. The composite of claim 1, wherein the transition metal catalyst is a photocatalyst.

7. The composite of claim 1, wherein the transition metal catalyst has electronic structure for energy transfer to a protein labeling agent.

8. The composite of claim 7, wherein the energy transfer is Dexter energy transfer.

9. The composite of claim 1, wherein the biomolecular condensate is selected from the group consisting of P-bodies, U-bodies, stress granules, centrosomes, signaling clusters, membrane clusters, synaptic densities, RNA transport granules, Balbiani bodies, germ granules, nuclear speckles, OPT domains, gems, PcG bodies, Cajal bodies, perinucleolar compartments, cleavage bodies, and PML bodies.

10. A system for profiling interactome of a biomolecular condensate comprising:
a protein labeling agent; and
a composite, the composite including one or more biomolecular condensates comprising nucleic acids and/or functionally diverse proteins, and a transition metal catalyst coupled to the biomolecular condensate via interaction with at least one of the nucleic acids or functionally diverse proteins, wherein the transition metal catalyst has electronic structure permitting energy transfer to the protein labeling agent to provide a reactive intermediate.

11. The system of claim 10, wherein the reactive intermediate is operable to label a protein or other biomolecule within a predetermined radius of the composite.

12. The system of claim 10, wherein the transition metal complex is of the formula: 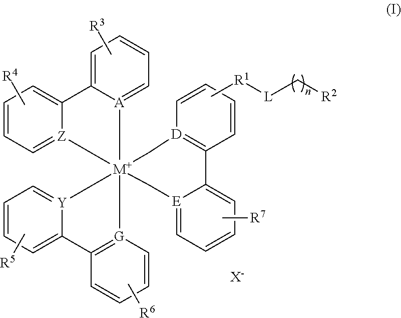
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X is a counterion, and n is an integer from 0 to 20.

13. The system of claim 12, wherein the transition metal complex is coupled to a protein of the biomolecular condensate via binding with haloalkane dehalogenase.

14. The system of claim 12, wherein the Mis iridium.

15. The system of claim 10, wherein the transition metal catalyst is a photocatalyst.

16. The system of claim 10, wherein the energy transfer is Dexter energy transfer.

17. The system of claim 10, wherein the protein labeling agent is a diazirine.

18. The system of claim 17, wherein the diazirine comprises a molecular marker.

19. The system of claim 10, wherein the biomolecular condensate is selected from the group consisting of P-bodies, U-bodies, stress granules, centrosomes, signaling clusters, membrane clusters, synaptic densities, RNA transport granules, Balbiani bodies, germ granules, nuclear speckles, OPT domains, gems, PcG bodies, Cajal bodies, perinucleolar compartments, cleavage bodies, and PML bodies.

20. A method of profiling microenvironments local to biomolecular condensates comprising:
forming a composite comprising a transition metal catalyst coupled to a biomolecular condensate via interaction with a biomolecular species of the biomolecular condensate;
activating a protein labeling agent to a reactive intermediate with the transition metal catalyst; and
coupling the reactive intermediate to a protein or other biomolecules within a predetermined radius of the composite.

21. The method of claim 20, wherein activating the protein labeling agent to the reactive intermediate comprises energy transfer from the transition metal catalyst to the protein labeling agent.

22. The method of claim 20, wherein the protein labeling agent is a diazirine.

23. (canceled)

24. The method of claim 20, wherein the predetermined radius is 2-100 nm.

25. (canceled)

26. (canceled)

27. The method of claim 20, wherein the protein labeling agent is activated at differing times in the biomolecular condensate lifecycle.

28. (canceled)

29. (canceled)

30. The method of claim 29, wherein the transition metal complex is coupled to a protein of the biomolecular condensate via binding with haloalkane dehalogenase.

31. (canceled)

32. (canceled)

33. The method of claim 20, wherein the biomolecular condensate is selected from the group consisting of P-bodies, U-bodies, stress granules, centrosomes, signaling clusters, membrane clusters, synaptic densities, RNA transport granules, Balbiani bodies, germ granules, nuclear speckles, OPT domains, gems, PcG bodies, Cajal bodies, perinucleolar compartments, cleavage bodies, and PML bodies.

34. (canceled)

35. (canceled)
